(19) United States
(12) Patent Application Publication
(10) Pub. No.: US 2025/0357004 A1
(43) Pub. Date: Nov. 20, 2025

(54) COMPUTING SYSTEMS AND METHODS FOR USING A PREDICTIVE MODEL AND HEALTH CONDITION DATA TO PREDICT A HEALTHCARE OUTCOME

(71) Applicant: UNIVERSITY OF MARYLAND, BALTIMORE COUNTY, Baltimore, MD (US)

(72) Inventor: Ian Stockwell, Cooksville, MD (US)

(21) Appl. No.: 19/200,870

(22) Filed: May 7, 2025

Related U.S. Application Data
(60) Provisional application No. 63/647,115, filed on May 14, 2024.

(51) Int. Cl. G16H 50/20; G16H 10/60; G16H 50/30

(52) U.S. Cl. CPC G16H 50/20; G16H 10/60; G16H 50/30

(57) ABSTRACT

Computing systems and methods for using a predictive model and health condition data to predict a healthcare outcome are disclosed. According to an aspect, a computing system include a healthcare data analyzer configured to receive a predictive model. The predictive model is adapted to use one or more inputs of a person's health data for predicting a healthcare outcome for the person. The healthcare data analyzer is configured to extract data of one or more risk factors from data indicative of the health condition of the person. The healthcare data analyzer is also configured to map the extracted data to the input(s) of the predictive model. Further, the healthcare data analyzer is configured to input the risk factor data into the predictive model to generate a prediction of the healthcare outcome for the person. The healthcare data analyzer presents the prediction of the healthcare outcome for the person.

### CROSS-REFERENCE TO RELATED APPLICATIONS

This application claims priority to U.S. Patent Provisional Application No. 63/647,115, filed May 14, 2024, and titled MODEL AGNOSTIC PREDICTION INFRASTRUCTURE, the content of which is incorporated herein by reference in its entirety.

### BACKGROUND

Advancing the health and welfare for patients is multifaceted and involves various stakeholders, including government regulatory agencies, healthcare providers, researchers, and community organizations. Electronic health records (EHRs), hospital admission records, and health information exchange systems improve the efficiency and coordination of healthcare services but are currently being outpaced by the evolving landscape of machine learning, data analytics, and artificial intelligence-based progress. Applied artificial intelligence (AI) and machine learning (ML) is rapidly changing healthcare, and will drive dramatic positive change for patients, their providers, and health systems. However, existing AI/ML solutions are currently fragmented and piecemeal, focusing on single conditions for a small group of potential users, without an underlying infrastructure bringing them together within a single platform.

Many talented researchers are building predictive and prescriptive models to identify the risk of a broad range of adverse health events (such as an unnecessary hospitalization, the onset of a preventable disease, or premature death) to drive important care interventions. However, such models may rarely be implemented due to the large financial, technical, and effort costs of moving models from the lab to production. In view of these considerations, there is a need to develop an infrastructure that can facilitate the use of this research in practice to thereby allow academic models to be deployed efficiently and effectively across health software platforms and data streams with minimal additional effort by researchers.

### SUMMARY OF THE DISCLOSURE

The presently disclosed subject matter relates to computing systems and methods for using a predictive model and health condition data to predict a healthcare outcome. According to an aspect, a computing system include a healthcare data analyzer configured to receive a predictive model. The predictive model is adapted to use one or more inputs of a person's health data for predicting a healthcare outcome for the person. The healthcare data analyzer is configured to receive data indicative of a health condition of a person. Further, the healthcare data analyzer is configured to extract data of one or more risk factors from the received data indicative of the health condition of the person. The risk factor(s) are associated with the healthcare outcome. The healthcare data analyzer is also configured to map the extracted data to the input(s) of the predictive model. Further, the healthcare data analyzer is configured to input the mapped, extracted risk factor data into the input(s) of the predictive model to generate a prediction of the healthcare outcome for the person. Further, the healthcare data analyzer is configured to present the prediction of the healthcare outcome for the person.

According to another aspect, a method for predicting a healthcare outcome includes receiving a predictive model. The predictive model is adapted to use one or more inputs of a person's health data for predicting a healthcare outcome for the person. The method also includes receiving data indicative of a health condition of a person. Further, the method includes extract data of one or more risk factors from the received data indicative of the health condition of the person. The risk factor(s) are associated with the healthcare outcome. The method also includes mapping the extracted data to the input(s) of the predictive model. Further, the method includes inputting the mapped, extracted risk factor data into the input(s) of the predictive model to generate a prediction of the healthcare outcome for the person. The method also includes presenting the prediction of the healthcare outcome for the person.

According to another aspect, a computing system include a healthcare data analyzer configured to receive a prescription model. The prescription model is adapted to use one or more inputs of a person's health data for prescribing an action for the person. The healthcare data analyzer is configured to receive data indicative of a health condition of a person. Further, the healthcare data analyzer is configured to extract data of one or more factors from the received data associated with a healthcare action. The factor(s) are associated with the healthcare outcome. The healthcare data analyzer is also configured to map the extracted data to the input(s) of the prescription model. Further, the healthcare data analyzer is configured to input the mapped, extracted risk factor data into the input(s) of the prescription model to generate an action for the person. Further, the healthcare data analyzer is configured to present the action of the healthcare outcome for the person.

According to another aspect, a method for predicting a healthcare outcome includes receiving a prescription model. The prescription model is adapted to use one or more inputs of a person's health data for predicting a healthcare outcome for the person. The method also includes receiving data indicative of a health condition of a person. Further, the method includes extract data of one or more factors from the received data indicative of the health condition of the person. The factor(s) are associated with a healthcare action. The method also includes mapping the extracted data to the input(s) of the predictive model. Further, the method includes inputting the mapped, extracted factor data into the input(s) of the prescription model to generate an action for the person. The method also includes presenting the action for the person.

### BRIEF DESCRIPTION OF DRAWINGS

Having thus described the presently disclosed subject matter in general terms, reference will now be made to the accompanying Drawings, which are not necessarily drawn to scale, and wherein:

FIG. 1 is a block diagram of a computing system for using a predictive model and health condition data to predict a healthcare outcome in accordance with embodiments of the present disclosure;

FIG. 2 is a flowchart of a method for using a predictive model and health condition data to predict a healthcare outcome in accordance with embodiments of the present disclosure;

FIG. 3 is a block diagram of a computing system for using a prescriptive model and health condition data to prescribe an action in accordance with embodiments of the present disclosure; and

FIG. 4 is a diagram depicting an example software stack for implementing the presently disclosed subject matter.

### DETAILED DESCRIPTION OF THE DISCLOSURE

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Unless otherwise defined, all technical terms used herein have the same meaning as commonly understood by one of ordinary skill in the art to which this disclosure belongs.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

As referred to herein, the terms “computing device” and “entities” should be broadly construed and should be understood to be interchangeable. They may include any type of computing device, for example, a server, a desktop computer, a laptop computer, a smart phone, a mobile phone, a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a GUI that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

As referred to herein, a computer network may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

As referred to herein, the term “predictive model” refers to a system or algorithm adapted to use data to predict an outcome and/or present factor associated with the outcome. The predictive model may be structured based on historical data to identify patterns. The predictive model can apply these patterns to input data to generate a prediction of an outcome or factor associated with the outcome. Example models include, but are not limited to, decision trees, regression models, and neural networks. In addition, a predictive model may be a large language model (LLM).

As referred to herein, the term “prescriptive model” refers to a system or algorithm adapted to use data to prescribe an action based on input data. The prescriptive model may be structured based on historical data to identify patterns. The prescriptive model can apply these patterns to input data to generate a prescribed action for a healthcare patient. Example models include, but are not limited to, decision trees, regression models, and neural networks. In addition, a prescriptive model may be a large language model (LLM).

As referred to herein, the term “health data” can refer to any data indicative of a state of health or a condition of health of a person. Health data can include, but is not limited to, vital signs, lab results, genomic data, mental health data, demographic data, symptoms, medical history data, lifestyle data, medications, and the like. Example vital signs include, but are not limited to, heart rate, blood pressure, respiratory rate, body temperature, and the like. Lab results include, but are not limited to, blood tests (e.g., cholesterol, glucose levels, etc.), urine tests, imaging results (e.g., X-rays, MRIs, and the like). Genomic data includes genetic profiles or predispositions to certain conditions. Mental health data includes diagnoses (e.g., depression or anxiety), therapy records, or stress levels. Demographic data includes, but is not limited to, age, sex, weight, height, BMI, and the like. Symptoms includes, but are not limited to, self-reported issues such as pain, fatigue, or fever. Medical history data include, but are not limited to, past diagnoses, surgeries, allergies, chronic conditions (e.g., diabetes, asthma), and the like. Lifestyle data includes, but is not limited to, diet, exercise habits, smoking status, alcohol consumption, and the like. Medications data include, but are not limited to, current or past prescriptions, dosages, supplements, and the like.

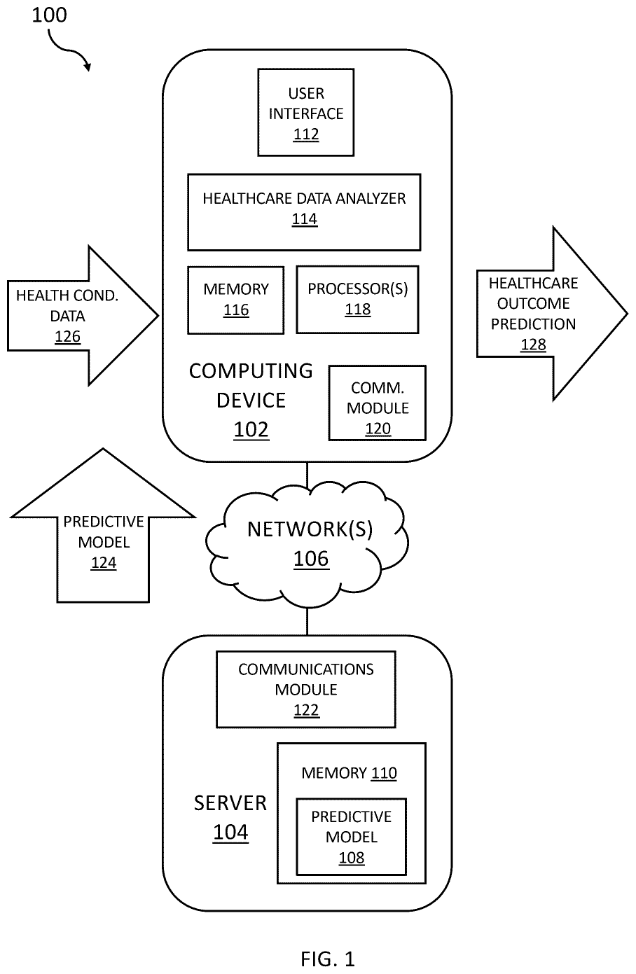

FIG. 1 illustrates a block diagram of a computing system 100 for using a predictive model and health condition data to predict a healthcare outcome in accordance with embodiments of the present disclosure. Referring to FIG. 1, the computing system 100 includes a computing device 102 and a server 104 that are communicatively connected via one or more networks 106. The server 104 is configured to store a predictive model 108 within its memory 110. The predictive model 108 is adapted to use one or more inputs of a person's health data for predicting a healthcare outcome for the person. For example, the predictive model 108 can be used to output a risk score and/or a risk factor based on inputs of a person's health data. The predictive model 108 can be any suitable predictive model trained in a research setting. Example health data for input into the predictive model 108 includes, but is not limited to, vital signs, lab results, genomic data, mental health data, demographic data, symptoms, medical history data, lifestyle data, medications, and the like. The predictive model 108 can be accessed by an authorized request to the server 104 via the network(s) 106.

At computing device 102, a user can interact with the computing device by use of a user interface 112. The user can suitably interact with the user interface 112 to utilize a healthcare data analyzer 114 in accordance with embodiments of the present disclosure. For example, the user may be a researcher or healthcare practitioner. As described in further detail herein, the healthcare data analyzer 114 may be an application residing on the computing device 102 for using a predictive model for predicting a healthcare outcome of a person, such as a patient. The user may use the user interface 112 to select the predictive model. Further, the user may interact with the user interface 112 to select the person's health condition data for input into the predictive model. Subsequent to entry of the person's health condition data into the predictive model, the predictive model can generate a prediction of the healthcare outcome for the person and present the prediction of the healthcare outcome for the system. For example, the user interface 112 may include a display for presenting the prediction information.

Functionalities of the healthcare data analyzer 114 described herein can be implemented by hardware, software, and/or firmware residing on the computing device 102. Alternatively, functionalities of the healthcare data analyzer 114 can be partially implemented by one or more other computing devices (e.g., server 104 or another computing device not shown in FIG. 1) that work in coordination with the computing device 102. The computing device 102 may include memory 116 and one or more processors 118 configured to implement functionalities of the healthcare data analyzer 114. For example, instructions for implementing these functionalities may be stored within memory 116, and the processor(s) 118 may implement the instructions.

In accordance with embodiments, the healthcare data analyzer 114 is configured to receive a predictive model that is adapted to use one or more inputs of a person's health data for predicting a healthcare outcome for the person. As an example, the predictive model can be used to generate a prediction of the healthcare outcome for a patient that results in actionable data for healthcare practitioners, healthcare organizations, or care managers that drive interventions for preventing adverse healthcare events or otherwise drive other actions for improving the patient's health. The information generated by the predictive model can be used for managing the patient to increase the likelihood of positive health outcomes. Further, users can prioritize high-risk patients and use the patient's unique risk profile to drive any needed clinical intervention. For example, output of the predictive model can be used for identifying which patients have the highest risk of developing diabetes and/or managing a patient's existing diabetes. Example adverse health conditions include, but are not limited to, dementia, mental health conditions, cardiovascular disease, and chronic kidney disease. Example adverse events include, but are not limited to, emergency department visits, hospital readmissions, and medication non-adherence.

During use of the healthcare data analyzer 114, the computing device 102 may suitably receive a predictive model. For example, a communication module 120 of the computing device 102 and a communication module 122 of the server 104 can operate together to communicate the communication of the predictive model 108 to the computing device 102 via network(s) 106. Arrow 124 depicts the communication of the predictive model 108 to computing device 102. The predictive model 108 can be stored in memory 116 of the computing device 102 or in another memory that is accessible by the computing device 102. The predictive model 108 is then thereby usable by a user at computing device 102.

The healthcare data analyzer 114 can receive data indicative of a health condition of a person. For example, the user can interact with the user interface 110 for selecting a patient's data indicative of a health condition. The user may select the data from an electronic health record, a hospital admission record, healthcare payment data, patient assessment data, or the like. This data may be stored locally within memory 116 of the computing device 102 or at another computing device accessible by the computing device 102.

Subsequent to receipt of the prediction model and data indicative of the health condition of the person, the healthcare data analyzer 114 can extract data of one or more risk factors from the data indicative of the health condition of the person. The risk factor(s) are associated with the healthcare outcome. For example, the risk factors can be data that indicates a metric or other indicator of a risk for hypertension, hyperlipidemia, diabetes, cardiovascular disease, or other adverse condition. These risk factors can be assigned using existing phenotyping methodologies, or through novel feature engineering methods.

The healthcare data analyzer 114 can map the extracted data to one or more inputs of the predictive model. Further, the healthcare data analyzer 114 can input the mapped, extracted risk factor data into the input(s) of the predictive model to generate a prediction of the healthcare outcome for the person. In addition, the system can use unmapped, model-specific risk factors when necessary. The healthcare data analyzer 114 can subsequently present the prediction of the healthcare outcome for the person. For example, the healthcare data analyzer 114 can use the user interface 112 to present the prediction data or information. As an example, the prediction data may be presented by text or graphically.

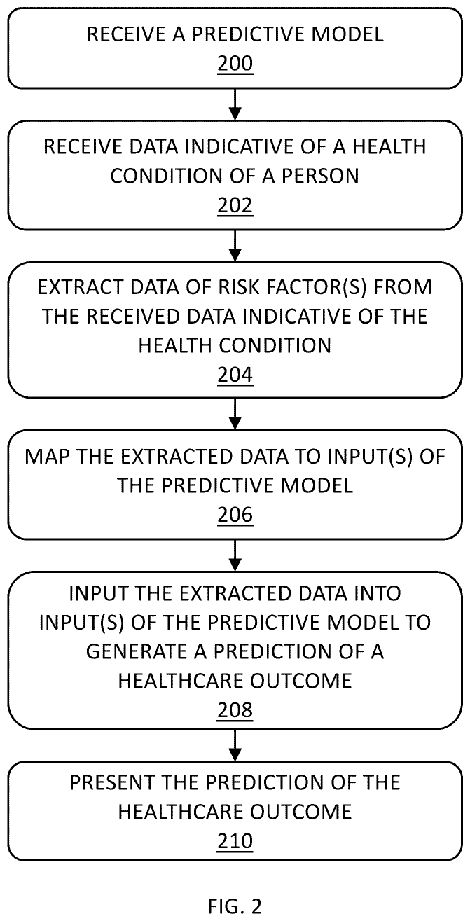

FIG. 2 illustrates a flowchart of a method for using a predictive model and health condition data to predict a healthcare outcome in accordance with embodiments of the present disclosure. The method is described by example as being implemented by the healthcare data analyzer 114 of the computing device 102 shown in FIG. 1, although it should be understood that the method may alternatively be implemented by another suitable computing device.

Referring to FIG. 2, the method includes receiving 200 a predictive model that is adapted to use one or more inputs of a person's health data for predicting a healthcare outcome for the person. For example, the healthcare data analyzer 114 can receive a predictive model 108 from server 104.

The method of FIG. 2 also includes receiving 202 data indicative of a health condition of a person. Continuing the aforementioned example, the healthcare data analyzer 114 can receive data indicative of a health condition of a person. Arrow 126 depicts receipt of health condition data at computing device 102.

The method of FIG. 2 includes extracting 204 data of one or more risk factors from the received data indicative of the health condition of the person, wherein the one or more risk factors are associated with the healthcare outcome. Continuing the aforementioned example, the healthcare data analyzer 114 can extract data of one or more risk factors from the received data indicative of the health condition of the person. The risk factors can be data that indicates a metric or other indicator of a risk for hypertension, hyperlipidemia, diabetes, cardiovascular disease, or other adverse condition.

The method of FIG. 2 includes mapping 206 the extracted data to the one or more inputs of the predictive model. Continuing the aforementioned example, the healthcare data analyzer 114 can map the extracted data to one or more inputs of the predictive model.

The method of FIG. 2 includes inputting 208 the mapped, extracted risk factor data into the one or more inputs of the predictive model to generate a prediction of the healthcare outcome for the person. Continuing the aforementioned example, the healthcare data analyzer 114 can input the mapped, extracted risk factor data into the input(s) of the predictive model to generate a prediction of the healthcare outcome for the person.

The method of FIG. 2 includes presenting 210 the prediction of the healthcare outcome for the person. Continuing the aforementioned example, the healthcare data analyzer 114 can use the user interface 112 to present the prediction data or information. Arrow 128 represents output of a healthcare outcome prediction that can be presented to a user.

In an example use case, the statistical power of predictive models can help health organizations prioritize patient care based on levels of risk, bring predictive machine learning solutions into the provider workflow, creating actionable data for healthcare organizations and care managers that drive interventions for preventing adverse health events. Users of healthcare data analyzers described herein can be care managers that operate under value-based care plans where clinical resources are limited and contracts financially reward healthcare organizations for avoiding adverse events. High-risk patients can be prioritized, and the patient's unique risk profile can be used to drive the any needed clinical intervention.

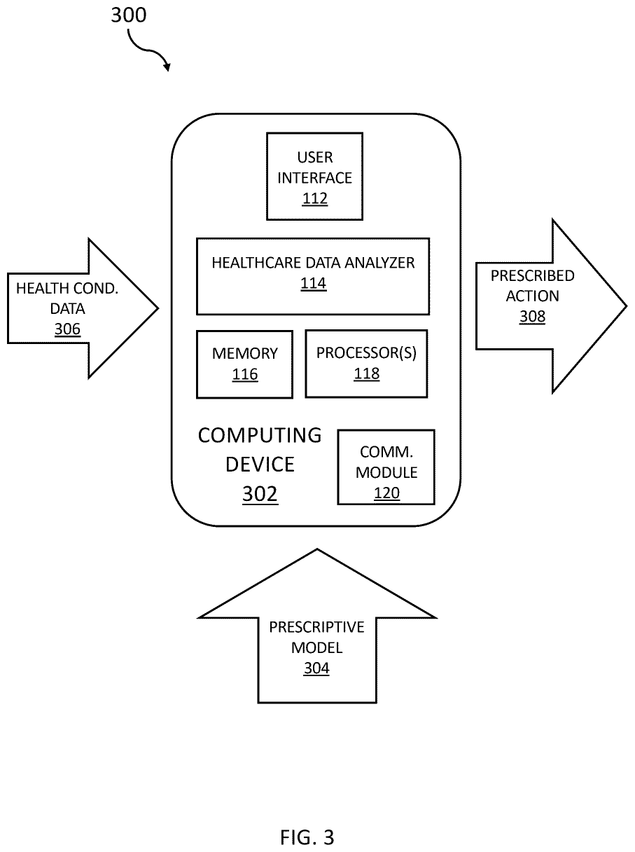

FIG. 3 illustrates a block diagram of a computing system 300 for using a prescriptive model and health condition data to prescribe an action in accordance with embodiments of the present disclosure. Referring to FIG. 3, the system 300 includes a computing device 302 similar to the computing device 102 shown in FIG. 1 except that the healthcare data analyzer is configured to utilize a predictive model for providing a prescribed action based on health condition data of a person.

Referring to FIG. 3, the computing device 102 can receive a prescriptive model 124. For example, the prescriptive model 304 can be received from another computing device. Further, the computing device 126 can receive health condition data 306 from another computing device or the data may be entered manually at the computing device 302. The prescriptive model 304 and data 306 may be stored within memory 116.

Further, the healthcare data analyzer 114 can extract data of one or more factors from the received data indicative of the health condition of the person. The factor(s) can be associated with a healthcare action. The healthcare data analyzer 114 can map the extracted data to the one or more inputs of the prescription model. Further, the healthcare data analyzer 114 can input the mapped, extracted factor data into the one or more inputs of the prescription model to generate an action for the person. The healthcare data analyzer 114 can also use the user interface 112 to present the prescribed action (e.g., indicated by arrow 308) for the person. As an example, the action is associated with a healthcare action for treating diabetes or cardiovascular disease. A user may have a panel of 1,000 patients under their care. 100 of these patients have reported signs of pre-diabetes. Therefore, a diabetes prediction model would allow users to rank those 100 patients by their likelihood of moving to full diabetes, and users could provide clinical guidance to all with direct outreach to those with the highest risk

In accordance with embodiments, healthcare data analyzers as described herein may utilize models of academically trained scoring algorithms for outcome prediction and prescription of action. In an example use case, models are scored across two domains: relevance and potential impact can be determined based on previous studies identifying high-burden adverse events and chronic conditions prevalent in the aging population. Statistical validity can be determined based on robustness of methodology, generalizability of the training sample, and predictive accuracy. Models may be ranked accordingly from highest potential impact to lowest. The prediction library may be expanded by adapting and integrating high-value models into the infrastructure. This can involve use of interfaces to map predictive features (e.g., hyperlipidemia and osteoporosis) to a data model and implementing a scoring algorithm within a production environment.

The presently disclosed subject matter can be compatible with Health Information Exchanges. This can involve use of APIs for secure data sharing, cloud computing for efficient processing, implementing version control, monitoring capabilities for deployed models, and use of real-time scoring pipelines that can interface with various healthcare data streams. Further, interfaces can present risk scores and supporting evidence in an actionable format. Success metrics can include both technical performance (e.g., prediction accuracy and system latency) and demonstration of compatibility with health information exchange standards (e.g., HL7/FHIR compliance, security requirements, and data exchange protocols).

In example use cases, the presently disclosed subject matter provides a platform for increasing the efficiencies of translating research or academically-trained AI/ML models into deployable healthcare solutions. Particularly, the presently disclosed subject matter can provide a standardized, reusable pathway. Example components include: a flexible feature extractor capable of ingesting and standardizing risk factors from disparate data sources (e.g., EHRs, claims, etc.), leveraging common data models such as OMOP where applicable; an adaptable algorithm library configured to host diverse predictive models regardless of their underlying statistical methodology; and integration-ready output mechanisms configured for compatibility with existing clinical IT systems, such as HIEs and EHRs.

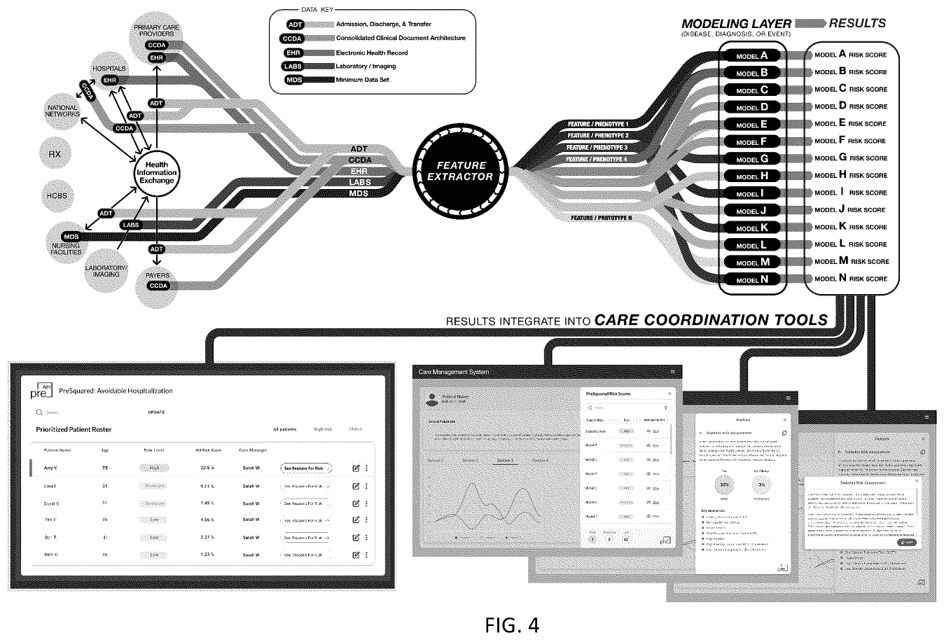

FIG. 4 illustrates a diagram depicting an example software stack for implementing the presently disclosed subject matter.

In embodiments, a healthcare data analyzer may be configured to handle multiple predictive models and/or prescriptive models. For example, the healthcare data analyzer 114 shown in FIG. 1 can present options for choosing among multiple different predictive models and/or prescriptive models. Identification of the different models may be presented via the ser interface 112. The models may reside in memory 116 of the computing device 102, or within memory of other computing devices, such as server 104. Upon selection of one of the models, the healthcare data analyzer 114 can use the selected predictive model or prescriptive model for predicting a healthcare outcome for a person or for prescribing an action for a person in accordance with embodiments disclosed herein.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a mobile computer with a smart phone client, or the like. A computing device can also include any type of conventional computer, for example, a laptop computer or a tablet computer. A typical mobile computing device is a wireless data access-enabled device (e.g., an iPHONE® smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

1. A computing system comprising:
a healthcare data analyzer configured to:
receive a predictive model that is adapted to use one or more inputs of a person's health data for predicting a healthcare outcome for the person;
receive data indicative of a health condition of a person;
extract data of one or more risk factors from the received data indicative of the health condition of the person, wherein the one or more risk factors are associated with the healthcare outcome;
map the extracted data to the one or more inputs of the predictive model;
input the mapped, extracted factor data into the one or more inputs of the predictive model to generate a prediction of the healthcare outcome for the person; and
present the prediction of the healthcare outcome for the person.

2. The computing system of claim 1, wherein the one or more risk factors include age, chronic condition data, environmental factors, or prior utilization.

3. The computing system of claim 1, wherein the healthcare data analyzer is configured to receive the data indicative of the health condition of the person from an electronic health record, a hospital admission record, healthcare payment data, or patient assessment.

4. The computing system of claim 1, wherein the predictive model outputs a risk score and/or a risk factor based on the input mapped, extracted risk factor data.

5. The computing system of claim 4, wherein the healthcare data analyzer configured to present the risk score and/or risk factor.

6. The computing system of claim 1, wherein the healthcare outcome is associated with an outcome for diabetes, dementia, mental health, chronic kidney disease, or cardiovascular disease.

7. The computing system of claim 1, wherein the one or more risk factors include a risk factor for conditions including hypertension, hyperlipidemia, diabetes, or cardiovascular disease.

8. The computing system of claim 1, wherein the healthcare data analyzer is configured to:
receive, via a user interface, selection of the predictive model among a plurality of models; and
initiate the extraction, mapping and inputting in response to receipt of the selection.

9. A method comprising:
receiving a predictive model that is adapted to use one or more inputs of a person's health data for predicting a healthcare outcome for the person;
receiving data indicative of a health condition of a person;
extracting data of one or more risk factors from the received data indicative of the health condition of the person, wherein the one or more risk factors are associated with the healthcare outcome;
mapping the extracted data to the one or more inputs of the predictive model;
inputting the mapped, extracted factor data into the one or more inputs of the predictive model to generate a prediction of the healthcare outcome for the person; and
presenting the prediction of the healthcare outcome for the person.

10. The method of claim 9, wherein the one or more risk factors include age, chronic condition data, or prior utilization.

11. The method of claim 9, further comprising receiving the data indicative of the health condition of the person from an electronic health record, a hospital admission record, or healthcare payment data.

12. The method of claim 9, wherein the predictive model outputs a risk score and/or a risk factor based on the input mapped, extracted risk factor data.

13. The method of claim 12, further comprising presenting the risk score and/or risk factor.

14. The method of claim 9, wherein the healthcare outcome is associated with an outcome for diabetes or cardiovascular disease.

15. The method of claim 9, wherein the one or more risk factors include a risk factor for hypertension, hyperlipidemia, diabetes, or cardiovascular disease.

16. The method of claim 9, further comprising:
receiving, via a user interface, selection of the predictive model among a plurality of models; and
initiating the extracting, mapping and inputting in response to receipt of the selection.

17. A computing system comprising:
a healthcare data analyzer configured to:
receive a prescription model that is adapted to use one or more inputs of a person's health data for prescribing an action for the person;
receive data indicative of a health condition of a person;
extract data of one or more factors from the received data indicative of the health condition of the person, wherein the one or more factors are associated with a healthcare action;
map the extracted data to the one or more inputs of the prescription model;
input the mapped, extracted risk factor data into the one or more inputs of the prescription model to generate an action for the person; and
present the action for the person.

18. The computing system of claim 17, wherein the one or more factors include age, chronic condition data, or prior utilization.

19. The computing system of claim 17, wherein the healthcare data analyzer is configured to receive the data indicative of the health condition of the person from an electronic health record, a hospital admission record, or healthcare payment data.

20. The computing system of claim 17, wherein the prescription model outputs a healthcare action to implement for the person.

21. The computing system of claim 17, wherein the action is associated with a healthcare action for treating diabetes or cardiovascular disease.

22. The computing system of claim 17, wherein the healthcare data analyzer is configured to:
receive, via a user interface, selection of the prescription model among a plurality of models; and
initiate the extraction, mapping and inputting in response to receipt of the selection.

23. A method comprising:
receiving a prescription model that is adapted to use one or more inputs of a person's health data for prescribing an action for the person;
receiving data indicative of a health condition of a person;
extracting data of one or more factors from the received data indicative of the health condition of the person, wherein the one or more factors are associated with a healthcare action;
mapping the extracted data to the one or more inputs of the prescription model;
inputting the mapped, extracted risk factor data into the one or more inputs of the prescription model to generate an action for the person; and
presenting the action for the person.

24. The method of claim 23, wherein the one or more factors include age, chronic condition data, or prior utilization.

25. The method of claim 23, further comprising receiving the data indicative of the health condition of the person from an electronic health record, a hospital admission record, or healthcare payment data.

26. The method of claim 23, wherein the prescription model outputs a healthcare action to implement for the person.

27. The method of claim 23, wherein the action is associated with a healthcare action for treating diabetes or cardiovascular disease.

28. The method of claim 23, further comprising:
receiving, via a user interface, selection of the prescription model among a plurality of models; and
initiating the extracting, mapping and inputting in response to receipt of the selection.
